(19) United States
(12) Patent Application Publication
(10) Pub. No.: US 2014/0363499 A1
(43) Pub. Date: Dec. 11, 2014

(54) USE OF A PARTICULATE IMMUNOMODULATOR IN CANCER THERAPY

(71) Applicant: EPITARGET AS, Oslo (NO)

(72) Inventor: Esben A. NILSSEN, Oslo (NO)

(21) Appl. No.: 14/470,406

(22) Filed: Aug. 27, 2014

Related U.S. Application Data
(63) Continuation of application No. PCT/NO2013/050037, filed on Feb. 27, 2013.

(30) Foreign Application Priority Data
Feb. 27, 2012 (NO) 20120211

(51) Int. Cl. A61K 9/127; A61K 47/10; A61K 47/24; A61K 38/19

(52) U.S. Cl. CPC A61K 9/127; A61K 38/193; A61K 47/10; A61K 47/24
USPC 424/450; 424/85.1

(57) ABSTRACT

The current invention is directed to a particulate or vesicular formulation of an immune modulating molecule, like e.g. cytokines, as well as uses, methods, compounds thereof. The formulation may be used to treat a range of diseases and conditions, in particular cancer.

### CROSS REFERENCE TO RELATED APPLICATIONS

This application is the Continuation of PCT International Application No. PCT/NO2013/050037, filed on Feb. 27, 2013, which claims priority under 35 U.S.C. 119(a) to Patent Application No. 20120211, filed in Norway on Feb. 27, 2012. This application also claims the benefit under 35 U.S.C. 119(e) to U.S. Provisional Application No. 61/610,669, filed on Mar. 14, 2012. All of which are hereby expressly incorporated by reference into the present application.

### FIELD OF THE INVENTION

The present invention is related to a particulate immunomodulator, like e.g. cytokines, for treating a range of conditions and disease states, in particular cancer, as well as uses, compositions, kits, and methods thereof.

### BACKGROUND OF THE INVENTION

White blood cells are involved in a variety of host defence mechanisms. Innate immune cells constitute a primary defence barrier against infectious agents while adaptive immunity provides a highly focused and powerful response. Cellular components of these responses involve a variety of leucocytes, including polymorphonuclear cells, monocytes and macrophages, and lymphocytes. These cells are also susceptible to participate in antitumor responses, although the development of tumours in a host is usually associated with a suppression of these potential cellular effectors. Suppression may be either non-specific, with a reduction of migration or phagocytic properties, or specific with deletion or inhibition of tumour-specific cells. A possibility for therapeutic intervention thus consists in the specific stimulation of cellular subtypes. The most classical is vaccination, which induces a highly targeted antigen-specific response. It is becoming increasingly clear that the global modulation of a leucocyte subpopulation could be of interest in the treatment of certain diseases such as cancer. Potential examples of this approach which are not yet applied in the clinic include the suppression of T regulatory cells which facilitate tumorigenesis or the stimulation of polymorphonuclear cells which are involved in so-called antibody dependent cellular cytotoxicity (ADCC) of therapeutic monoclonal antibodies (mAbs).

Growth factors, like e.g. the cytokine granulocyte-colony stimulating factor (G-CSF), are currently used in medical therapy to decrease the impacts of chemotherapy-induced neutropenia. In vitro studies suggest that cytokines like e.g. granulocyte-colony stimulating factor (G-CSF), granulocyte macrophage colony stimulating factor (GM-CSF), and interleukin-2 (IL-2) have a favourable effect on cancer in combination with monoclonal anibodies. See e.g. Honsik at al. (1986); Munn and Cheung (1987); Kushner and Cheung (1989); Ottonello et al. (1999); Stockmeyer et al. (2001); van der Kolk et al. (2002).

However, fast clearance and nonspecific biodistribution in vivo limits clinical use of cytokines. For example, G-CSF (filgrastim) has an elimination half-life of only 3.5 hrs. in humans. There are several approaches to improve the pharmacokinetics and modify the biodistribution of cytokines, including e.g. conjugation to polyethylene glycol (PEG; pegylation) or albumin, as well as encapsulation into particles or vesicles. Pegylation of filgrastim has been particularly successful, increasing the elimination half-life from 15 to 80 hrs. Another interesting approach has been liposomal encapsulation of cytokines. The focus areas of the liposome protagonists have typically been reduced biodistribution to irrelevant tissues, plasma clearance, and toxicity. Use of particulate targeting to leucocyte subpopulations would be of great value since it would allow both the use of considerably smaller doses and a reduced exposure of non-target tissues. Particulate distribution of therapeutic agents has been validated in a number of instances, for example in the case of liposomal anticancer agents such as doxorubicin or potentially nephrotoxic antimycotic agents such as amphotericin B.

Debs and coworkers (Debs et al. 1990) report liposomal Tumour Necrosis Factor a (TNF-α). The liposomes may comprise lipids phosphatidylserine (PS), phosphatidylglycerol (PG), or phosphatidylcholine (PC). All liposomes comprise cholesterol and a heterogeneous size distribution with a mean diameter of 2.03 μm is reported. No therapeutic advantage of liposomal TNF-α compared to free TNF-α was recorded.

Anderson and co-workers (Anderson et al. 1990) describe multilamellar vesicles consisting of DMPC, DMPG, and interleukine-2 (IL-2) for treatment of sarcoma pulmonary metastases.

Furthermore, Anderson and co-workers (Anderson et al. 1994) disclose liposomal cytokines comprising dimyristoylphosphatidylcholine (DMPC) and one of the cytokines IL-1, IL-2, IL-6, GM-CSF, or IFN-γ.

Meyer and coworkers (Meyer et al. 1994) report liposomal G-CSF comprising the phospholipids dimyristoylphosphatidylglycerol (DMPG), DMPC, and cholesterol.

Rourke and coworkers (Rourke et al. 1996) disclose liposomal formulations of G-CSF, pSt, IL-2, IL-4, and GM-CSF comprising one of the phospholipids DMPG, dipalmitoylphosphatidylglycerol (DPPG), dioleoylphosphatidylglycerol (DOPG), DMPS, DOPS, DMPC, DPPC, DOPC.

Cabanes and coworkers (Cabanes et al. 1999) disclose two liposomal formulations of IL-2 comprising either dimyristoyl phosphatidylcholine (DMPC) or egg phosphatidylcholine (EPC) as main lipids. Non of the formulations show improved therapeutic effect compared to untreated control.

Kedar and co-workers disclose a range of different liposomal formulations of IL-2, GM-CSF, TNF-α through four publications (Kedar et al. 1994a; Kedar et al. 1994b; Kedar et al. 1997; Kedar et al. 2000). In the last paper of the series it is concluded that large multilamellar vesicles consisting of DMPC and without steric stabilisation, that is, without polyethylene glycol (PEG), have a stronger immunomodulatory activity than sterically stabilised small egg PC based liposomes.

U.S. Pat. No. 5,225,212 (Martin et al.) mention liposomal formulations of polypeptides, including TNF, M-CSF, G-CSF, GM-CSF. The liposomes may comprise vesicle forming lipids phosphatidylcholine (PC), phosphatidylethanolamine (PE), phosphatidic acid (PA), phosphatidylinositol (PI), as well as PEG derivatised to phospholipids. However, the examples do not disclose formulations comprising underivatised PE or PS, in particular unsaturated PE or PS, and immunostimulatory agents, less that these formulations have favourable therapeutic effects on cancer.

None of the publications above mention or suggest the added therapeutic benefit of using unsaturated PE or phosphatidylserine (PS) based liposomes and/or high PEG concentrations in liposomal cytokines.

WO 96/29989 (Collins & Brems) discloses G-CSF:phospholipid complexes comprising negatively charged and unsaturated phospholipids. Phoshatidylethanolamine and phosphatidylserines are mentioned as possible phospholipids, although it is proved that phosphatidylglycerols (PG) are most suitable. Furthermore, PEG and cholesterol are not included in the complex and complex size is not controlled.

The current applicant has earlier disclosed liposomes comprising unsaturated phospholipids comprising anti-cancer peptides or proteins, like filgrastim, pegfilgrastim, or sargramostim (WO 2009/075582, WO 2010/143969, WO 2010/143970). The therapeutic use of such liposomal cytokines without acoustic triggering is not mentioned or suggested.

In the present disclosure, it is documented that the particle of the invention has a strong propensity to accumulate in lymphatic tissue, like e.g. spleen and lymph nodes. This characteristic is used to deliver immunomodulating substances, in particular, cytokines, to the immune apparatus of an animal or human being.

Moreover, the current applicant has found that certain particulate or vesicular formulations of cytokines have, even at very low cytokine concentrations, an improved effect on growth inhibition of tumours compared to free cytokines. Thus, the present invention may be used to improve cancer therapy alone or in combination with other treatment regimens.

### DEFINITIONS

DOPE herein means 1,2-Dioleoyl-sn-Glycero-3-Phosphoethanolamine

DSPC means 1,2-distearoyl-sn-glycero-3 phosphocholine or, in short, distearoylphosphatidylcholine.

DSPE means 1,2-distearoyl-sn-glycero-3-phosphoethanolamine or distearoylphosphatidylethanolamine.

DSPE-PEGXXXX means 1,2-distearoyl-sn-glycero-3-phosphoethanolamine-N-[methoxy(polyethylene glycol)-XXXX, wherein XXXX signifies the molecular weight of the polyethylene glycol moiety, e.g. DSPE-PEG2000 or DSPE-PEG5000.

IS herein mean Inverted Structure.

n-alcohol means any alcohol with n carbon atoms.

PC herein means phosphatidylcholine with any composition of acyl chain.

PE means phosphatidylethanolamine with any composition of acyl chain length.

PEG means polyethylene glycol or a derivate thereof.

PEGXXXX means polyethylene glycol or a derivate thereof, wherein XXXX signifies the molecular weight of the polyethylene glycol moiety.

POPE herein means 1-palmitoyl-2-oleoyl-sn-glycero-3-phosphoethanolamine.

SOPE herein means 1-stearoyl-2-oleoyl-sn-glycero-3-phosphoethanolamine.

‘Immunomodulator’ herein means a substance which influences either the absolute number or functions of leucocytes or a certain subpopulation of leucocytes.

### General Provisions

The phospholipid, cholesterol, PEG-lipid concentrations mentioned herein are nominal values unless stated otherwise.

In the current disclosure singular form means singular or plural. Hence, ‘a particle’ may mean one or several particles. Furthermore, all ranges mentioned herein includes the endpoints, e.g. the range ‘from 14 to 18’ includes 14 and 18.

### DETAILED DESCRIPTION OF THE INVENTION

The current inventors have found that certain particulate or vesicular formulations of immunomodulators improves the therapeutic outcome in diseased animals, in particular in animals with cancer.

Accordingly, the current invention relates to a particulate or vesicular material comprising an immunomodulator and a phospholipid.

The invention further relates to the particulate or vesicular material of the invention for use as a medicament.

The invention further relates to the particulate or vesicular material of the invention for use in treatment of a condition or a disease.

Yet another aspect of the invention is a composition or a pharmaceutical composition comprising the particulate or vesicular material of the invention.

A further aspect of the current invention is use of the particulate or vesicular material of the invention to manufacture a medicine for treating a disease or a condition, wherein said material if not activated by means of acoustic energy or ultrasound

A further aspect of the invention is a kit comprising the particulate or vesicular material of the current invention.

The invention also relates to a method of producing the composition or kit supra.

Another aspect of the current invention is a therapeutic or medical method for treating a disease or a condition comprising the steps of administering the particulate or vesicular material of the invention to a patient in need thereof.

Another aspect of the invention is directed to a method of treating a disease or a condition including administering to a subject in need thereof a composition including the particulate or vesicular material of the invention.

Another aspect of the invention is directed to a method of treating cancer including administering to a subject in need thereof a pharmaceutical composition including a liposome encapsulating a cytokine, wherein the liposome includes an unsaturated phoshatidylethanolamine (PE) or phosphatidylserine (PS), cholesterol, and polyethylene glycol (PEG) or a derivate thereof, and wherein the composition is not activated by acoustic energy or ultrasound.

A further aspect of the invention is directed to a pharmaceutical composition including a liposome encapsulating granulocyte-colony stimulating factor (G-CSF), wherein the liposome includes at least 52 mol % of 1,2-Dioleoyl-sn-Glycero-3-Phosphoethanolamine (DOPE).

The invention also relates to a method of producing the combination, composition, or kit.

Activation of, or triggered release from, the present particulate or vesicular material by means of acoustic energy or ultrasound is not part of the current invention. Furthermore, the present material is preferably not activated or triggered by heat.

The particulate or vesicular material or formulation may be arranged in any form of dispersion of a given internal structure. Examples of preferred structures are hexagonal structures (e.g. Hexosome®), cubic structures (e.g. Cubosomes®), emulsion, microemulsions, micelles, liquid crystalline particles, or liposomes. According to a preferred embodiment, the particulate material is a membrane structure, more preferably a liposome. A liposome normally consists of a lipid bilayer with an aqueous interior. Preparation of liposomes is well known within the art and a number of methods may be used to prepare the current material.

Said particulate or vesicular material or formulation may further comprise any lipid. Preferably, the lipid is an amphiphilic lipid such as a sphingolipid and/or a phospholipid. In a preferred embodiment the amphiphilic lipids are phospholipids of any type or source.

The phospholipids may be saturated or unsaturated, or a combination thereof, however, the phospholipids are preferably unsaturated. Typically, the selected phospholipids will have an acyl chain length at least 12 carbon atoms, more often at least 14 carbon atoms, and more often at least 16 carbon atoms, and even more often at least 18 carbon atoms. Preferably the acyl chain length is within the range 14 to 24 carbon atoms, more preferably 14 to 22 carbon atoms, even more preferably within 16 to 22 carbon atoms, even more preferably within 16 to 18. Acyl chain of different lengths may be mixed in the material of the invention, including asymmetric phospholipids, or all acyl chains may have similar or identical length. In a preferred embodiment of the current invention the length of both acyl chains of the phospholipid is 18 carbon atoms.

Furthermore, the polar head of the main phospholipid may be of any type, e.g. phosphatidylethanolamine (PE), phosphatidylcholine (PC), phosphatidic acid (PA), phosphatidylserine (PS), phosphatidylglycerol (PG), phosphatidylinositol. Also, the material or formulation may comprise mixtures of phospholipids with different polar heads. However, the lipids or phospholipids may not be derivatised to polyethylene glycol, like in e.g. DSPE-PEG, unless explicitly stated. At last one acyl chain may be unsaturated, however, it is preferred that both acyl chains are unsaturated. Lysolipids, like lysoPE, may also be included in the material of the invention. The phospholipid is preferably PE, PC, and/or PS, even more preferably unsaturated PE, PC, and/or PS, even more preferably unsaturated PE and/or PS. Preferred PEs are listed in Table 1 and 2, while preferred PCs are listed in Table 3 and 4. PS is preferably 1,2-dioleoyl-sn-glycero-3-phospho-L-serine (DOPS) or 1-stearoyl-2-oleoyl-sn-glycero-3-phospho-L-serine (SOSE), or a combination thereof. In embodiments of the current invention the phospholipid is 1,2-Dioleoyl-sn-Glycero-3-Phosphoethanolamine (DOPE), 1,2-Dioleoyl-sn-Glycero-3-Phosphoserine (DOPS), 1,2-Dioleoyl-sn-Glycero-3-Phosphocholine (DOPC), or hydrogenated soy PC. In a preferred embodiment of the current invention the phospholipid is DOPE.

The phospholipid concentration in the material or formulation of the current invention may be of any suitable level. Typically, the PC concentration will be within the range 50 to 80 mol %, preferably within the range 50 to 60 mol %. In one embodiment of the invention the PC concentrations, more specifically, the HSPC concentration, is about 57 mol %. However, if the PC is unsaturated, higher concentrations of PC are preferred. The PE or PS concentration should preferably be within the range 25 to 98 mol %, more preferably within the range 32 to 98 mol %, even more preferably 32 to 75 mol %, even more preferably 40 to 75 mol %, even more preferably 50 to 75 mol %, even more preferably 52 to 75 mol %. Medium to higher PE concentrations are preferred, e.g. at least 40 mol %, more preferably at least 50 mol %, more preferably at least 60 mol % PE. Accordingly, in preferred embodiments of the current invention the PE concentration is about 54, 58, or 62 mol %, while the PS concentration is 62 mol %.

TABLE 1
Symmetric PE

| Carbon number | Product |
|---|---|
| 16:0[(CH3)4] | Diphytanoyl PE |
| 16:1 | Dipalmitoleoyl PE |
| 18:1(delta 9-cis) | Dioleoyl (DOPE) |
| 18:1(delta 9-trans) | Dielaidoyl |
| 18:2 | Dilinoeoyl |
| 18:3 | Dilinolenoyl |
| 20:4 | Diarachidonoyl |
| 22:6 | Docosa-hexaenoyl |

TABLE 2
Asymmetric PE

| Carbon number | 1-Acyl | 2-Acyl |
|---|---|---|
| 16:0-18:1 | Palmitoyl | Oleoyl (POPE) |
| 16:0-18:2 | Palmitoyl | Linoleoyl |
| 16:0-20:4 | Palmitoyl | Arachidonoyl |
| 16:0-22:6 | Palmitoyl | Docosahexaenoyl |
| 18:0-18:1 | Stearoyl | Oleoyl (SOPE) |
| 18:0-18:2 | Stearoyl | Linoleoyl |
| 18:0-20:4 | Stearoyl | Arachidonoyl |
| 18:0-22:6 | Stearoyl | Docosahexaenoyl |

TABLE 3
Symmetric PC

| Carbon number | Trivial | IUPAC |
|---|---|---|
| 16:1 | Dipalmitoleoyl PC | 9-cis-hexadecenoyl PC |
| 18:1 | Petroselinoyl PC | 6-cis-octadecenoic PC |
| 18:1 | Oleoyl PC (DOPC) | 9-cis-octadecenoic PC |
| 18:1 | Elaidoyl PC | 9-trans-octadecenoic PC |
| 18:2 | Linoleoyl PC | 9-cis-12-cis- |
| | | octadecadienoic PC |
| 18:3 | Linolenoyl PC | 9-cis-12-cis-15- |
| | | cisoctadecatrienoic PC |
| 20:1 | Eicosenoyl PC | 11-cis-eicosenoic PC |
| 20:4 | Arachidonoyl PC | 5,8,11,14(all -cis) |
| | | eicosatetraenoic PC |
| 22:1 | Erucoyl PC | 13-cis-docosenoic |
| 22:6 | DHA PC | 4,7,10,13,16,19 (all -cis) |
| | | docosahexaenoic PC |
| 24:1 | Nervonoyl PC | 15-cis-tetracosenoic PC |

TABLE 4
Asymmetric PC

| Carbon Number | 1-Acyl | 2-Acyl |
|---|---|---|
| 18:0-18:1 | Stearoyl | Oleoyl |
| 18:0-18:2 | Stearoyl | Linoleoyl |
| 18:0-20:4 | Stearoyl | Arachidonoyl |
| 18:0-22:6 | Stearoyl | Docosahexaenoyl |

TABLE 5
PC

| Carbon Number | 1-Acyl | 2-Acyl |
|---|---|---|
| 18:0-18:1 | Stearoyl | Oleoyl |
| 18:0-18:2 | Stearoyl | Linoleoyl |
| 18:0-20:4 | Stearoyl | Arachidonoyl |
| 18:0-22:6 | Stearoyl | Docosahexaenoyl |

Components or stabilising agents for improving blood circulation time and/or further modulate efficacy, improve shelf life, etc, may be included in the material, like e.g. poly(oxazoline), polyvinyl alcohol, poly (glycerol), poly-N-vinylpyrrolidone, poly[N-(2-hydroxypropyl)methacrylamide], poly(amino acid)s, dextran, polyethylene glycol (PEG), or polymers. More specifically, the material or formulation may comprise e.g. polyvinyl alcohols, polyethylene glycols (PEG), dextrans, or other polymers or derivates thereof conjugated or associated to a molecule, e.g. a lipophilic molecule, to obtain anchoring to the current particulate material. PEG or a derivate thereof, at any suitable concentration, is preferred. An example of a PEG derivative would be 1,2-distearoyl-sn-glycero-3-phosphoethanolamine-N-[methoxy(polyethylene glycol)-2000 (DSPE-PEG200). However, PEG concentrations are preferably at least 2 mol %, more preferably at least 5 mol %, even more preferably at least 8 mol %, within the range 3 to 20 mol %, even more preferably within the range 4 to 20 mol %, and even more preferably within the range 8 to 20 mol %. In embodiments of the current invention the PEG concentration is 5, 8, 12, or 16 mol %. 8, 12, or 16 mol % are preferred, and the range 8 to 16 mol % is consequently particularly preferred. The PEG moiety may be of any molecular weight or type, however, it is preferred that the molecular weight is within the range 100 to 5000 Da, more preferably within 350 to 5000 Da, even more preferably within 2000 to 5000 Da. In a preferred embodiment the molecular weight is 2000 or 5000 Da. The PEG moiety may be associated with any molecule allowing it to form part of the particulate or vesicular material. Preferably, the PEG moiety is conjugated to a sphingolipid (e.g. ceramide), a glycerol based lipid (e.g. phospholipid), or a sterol (e.g. cholesterol), more preferably to a ceramide and/or PE, and even more preferably to PE, like DMPE, DPPE, or DSPE. The lipid-grafted PEG is preferably 1,2-distearoyl-sn-glycero-3-phosphoethanolamine-N-[methoxy(polyethylene glycol)-2000] (DSPE-PEG 2000) and/or 1,2-distearoyl-sn-glycero-3-phosphoethanolamine-N-[methoxy(polyethylene glycol)-5000] (DSPE-PEG 5000). In preferred embodiments of the current invention the lipid-grafted PEG is DSPE-PEG 2000 or DSPE-PEG5000.

The particulate material may also comprise a sterol. The particulate material may comprise any suitable sterol concentration, preferably cholesterol, depending on the specific particle properties. In general, 50 mol % sterol is considered the upper concentration limit in liposome membranes. The current particulate material preferably comprises up to 20 mol % cholesterol, more preferably up to 30 mol %, and even more preferably up to 40 mol % cholesterol, more preferably cholesterol concentrations within the range 1-40 mol %, more preferably 10-40 mol %, even more preferably 20-40 mol %, and most preferably within the range 30 to 40 mol %. In preferred embodiments of the current invention the particulate material comprises 20, 30, 38, or 40 mol % cholesterol. Accordingly, the cholesterol concentration is preferably within the range constituted by any of the mentioned embodiment concentrations.

In preferred embodiments the material has the composition DOPE:DSPE-PEG2000:Chol (mol %) 62:8:30, 58:12.30, 54:16:30, or 72:8:20.

The particulate material of the invention may be of any suitable size. However, the material should preferably have an average diameter (as measured by dynamic light scattering) within the range 50 to 2000 nm, more preferably with in the range 50 to 1200 nm, even more preferably within the average size range 80 to 800 nm, even more preferably within the range 80 to 400 nm, even more preferably within the range 80 to 200 nm. In embodiments of the current invention the average size is typically within the ranges 80 to 510 nm. The size distribution may be narrow or wide. Typically, all particulate material should be within the range 50 to 1500 nm.

Any immunomodulator may be associated with the current formulations. The association may be through strong chemical bonds, like e.g. covalent bonds, or weak bond, or hydrophilic or lipophilic interactions. For example, the cytokine may be conjugated to DSPE-PEG via a maleimide moiety. It is preferred that the association between immunomodulator and the current material is of non-covalent or weak nature, e.g. hydrophilic or lipophilic interactions. An ‘immunomodulator’ is defined as a substance which influences either the absolute number or functions of leucocytes or a certain subpopulation of leucocytes. The immunomodulator may be any immunomodulatory molecule, preferably a peptide or a protein and is, more preferably a cytokine. Examples of cytokines are colony-stimulating factor (CSF), interferon (IFN), interleukin (IL), stem cell factor (SCF), tumour growth factors (TGF), and tumour necrosis factor (TNF). Preferably, the cytokine is a CSF, IL, IFN, or any combination thereof; more preferably, the cytokines are CSF and/or IL; and most preferred the cytokine is a CSF. The CSF may be chosen from any one or any combination of the CSFs ancestim, garnocestim, pegacaristim, leridistim, milodistim, filgrastim, lenograstim, nartograstim, pegfilgrastim, pegnartograstim, ecogramostim, molgramostim, regramostim, sargramostim, cilmostim, lanimostim, mirimostim, daniplestim, muplestim, or derivates thereof.

The CSF may be a G-CSF, M-CSF, and/or GM-CSF, although G-CSF and GM-CSF are preferred. Any type of G-CSF or GM-CSF may be used alone or in combination, like e.g. filgrastim, lenograstim, nartograstim, pegfilgrastim, pegnartograstim, ecogramostim, molgramostim, regramostim, sargramostim, and/or derivates thereof, although, the G-CSFs filgrastim or lenograstim are preferred. In a preferred embodiments of the current invention the G-CSF is filgrastim or lenograstim. The interleukin may be of any sort and source. At present at least 35 major interleukins have been identified named from IL-1 to IL-35. Preferably the IL is IL-2 and/or IL-4, most preferably IL-2 like aldesleukin or a derivate thereof. An example of a derivative of aldesleukin would be PEG-aldesleukin.

The concentration of cytokine in the material of the invention may vary according to the therapeutic goals. For example, free G-CSF is generally dosed at 5-10 μg/kg/day for an expected duration of 14 days. In a mouse (20 g) this corresponds to a dose of approximately 60-120 μg/kg/day, or a total weekly dose of approx. 840 μg/kg. The current inventors have shown that a weekly dose of cytokine (including both liposomal and extraliposomal cytokine) as low as 37.5 μg/kg when formulated as herein described is superior to a weekly dose of 900 μg/kg of free G-CSF. Accordingly, the lipid/cytokine concentration ratio should be at least 10,000. By way of example, if the lipid concentration of a liposome (including phospholipids, PEG phospholipids, and cholesterol) is 30 mg/ml, then the cytokine concentration should be at least 3 μg/ml. In one embodiment of the current invention the nominal lipid/cytokine ratio is 4,000.

The particulate material of the invention may further comprise or be combined with any other drug or a functional molecule. The drug may be any drug suitable for the purpose. However, anti-bacterial drugs, anti-inflammatory drugs, immunosuppressive drugs, anti cancer drugs, or any combination thereof are preferred. As the current technology is particularly adapted for treating cancer, anti cancer drugs are preferred. Anti cancer drugs includes any chemotherapeutic, cytostatic or radiotherapeutic drug. It may be of special interest to load the current particulate material with oligonucleotides, more specifically deoxyribonucleic acid (DNA) or ribonucleic acid (RNA), in particular small interfering RNA (siRNA), microRNA, or antisense RNA.

The general groups of cytostatics are alkylating agents (L01A), anti-metabolites (L01 B), plant alkaloids and terpenoids (L01C), vinca alkaloids (L01 CA), podophyllotoxin (L01 CB), taxanes (L01 CD), topoisomerase inhibitors (L01CB and L01XX), antitumour antibiotics (L01D), platinum compounds, recombinant enzymes, hormonal therapy. Examples of cytostatics are gemcitabine, daunorubicin, cisplatin, docetaxel, 5-fluorouracil, vincristine, methotrexate, cyclophosphamide, L-asparaginase and doxorubicin.

Accordingly, the drug may include alkylating agents, antimetabolites, anti-mitotic agents, epipodophyllotoxins, antibiotics, hormones and hormone antagonists, enzymes, platinum coordination complexes, anthracenediones, substituted ureas, methylhydrazine derivatives, imidazotetrazine derivatives, cytoprotective agents, DNA topoisomerase inhibitors, biological response modifiers, retinoids and arsenic derivatives, therapeutic antibodies, differentiating agents, immunomodulatory agents, and angiogenesis inhibitors.

The drug may also be alpha emitters like e.g. radium-223 (223Ra) and/or thorium-227 (227Th) or beta emitters like yttrium-90. Other alpha emitting isotopes currently used in preclinical and clinical research include astatine-211 (211At), bismuth-213 (213Bi), and actinium-225 (225Ac).

Moreover, the drug may further comprise anti-cancer peptides, like telomerase or fragments of telomerase, like hTERT; or proteins, like e.g. monoclonal or polyclonal antibodies, scFv, tetrabodies, Vaccibodies, Troybodies, etc. Also, the material of the invention may comprise collagenases or other enzymes targeting the microenvironmental stroma, tumour endothelium, or surface antigens of tumour cells, particular proteins or molecules improving the uptake and distribution of particulate material in target tissues.

More specifically, therapeutic agents that may be included in the particulate material include abarelix, alemtuzumab, alitretinoin, allopurinol, altretamine, amifostine, anastrozole, arsenic trioxide, asparaginase, BCG live, bexarotene, bleomycin, bortezomib, busulfan, calusterone, camptothecin, capecitabine, carboplatin, carmustine, celecoxib, chlorambucil, cinacalcet, cisplatin, cladribine, cyclophosphamide, cytarabine, dacarbazine, dactinomycin, darbepoetin alfa, daunorubicin, denileukin diftitox, dexrazoxane, docetaxel, doxorubicin, dromostanolone, Elliott's B solution, epirubicin, epoetin alfa, estramustine, etoposide, exemestane, floxuridine, fludarabine, fluorouracil, fulvestrant, gemcitabine, gemtuzumab ozogamicin, gefitinib, goserelin, hydroxyurea, ibritumomab tiuxetan, idarubicin, ifosfamide, imatinib, interferon alfa-2a, interferon alfa-2b, irinotecan, letrozole, leucovorin, levamisole, lomustine, meclorethamine, megestrol, melphalan, mercaptopurine, mesna, methotrexate, methoxsalen, methylprednisolone, mitomycin C, mitotane, mitoxantrone, nandrolone, nofetumomab, oblimersen, oprelvekin, oxaliplatin, paclitaxel, pamidronate, pegademase, pegaspargase, pemetrexed, pentostatin, pipobroman, plicamycin, polifeprosan, porfimer, procarbazine, quinacrine, rasburicase, streptozocin, talc, tamoxifen, tarceva, temozolomide, teniposide, testolactone, thioguanine, thiotepa, topotecan, toremifene, tretinoin, uracil mustard, valrubicin, vinblastine, vincristine, vinorelbine, zoledronate, and elaidic acid ester of cytarabine.

Drugs may be modified by addition of a lipid. Such lipophilic drug should preferably comprise a long hydrocarbon chain and/or a hydrophobic ring structure. The hydrocarbon chain of the lipophilic drug is preferably at least 18 carbon atoms long. Preferably the hydrocabon chain is an elaidic acid. Most preferably, the lipophilic drug is an elaidic acid ester of gemcitabine, cytarabine, betamethason, prednisolon, acyclovir, ganciclovir, or ribavirin.

Furthermore, the particle of the invention may also comprise an imaging contrast agent, like e.g. an MR, X-ray, or optical imaging contrast agent, to render tracking and monitoring possible or a gamma-emitter for nuclear imaging. Examples of MR and X-ray contrast agents, as well as fluorescent and bioluminescent probes may be found in the literature.

The vesicle or particle of the invention does preferably not comprise any bubbles or undissolved gases, like e.g. found in microbubbles.

The particulate material of the invention may be used to treat a range of diseases and conditions. The condition or disease is preferably asthma, allergy, cancer, cardiovascular disease, autoimmune disorders, transplant rejection, infectious diseases, inflammatory diseases, degenerative diseases, haematological diseases, myalgic encephalopathy, chronic fatigue syndrome, post viral fatigue syndrome; more preferably, cancer, cardiovascular disease, or autoimmune disorders. In a preferred embodiment of the current invention the disease or condition is cancer. The cancer is preferably lymphoma, breast cancer, or colon cancer. In an embodiment of the current invention the lymphoma is a follicular non-Hodgkin's lymphoma.

As used herein a subject includes a bird, reptile, amphibian or mammal, such as a human, or a pet, for example, a cat or a dog, a farm animal, such as a pig, sheep, goat, horse, or cow.

### BRIEF DESCRIPTION OF THE DRAWINGS

FIG. 1. Optical imaging of mice treated with labelled liposomes up to 48 hr post inejction. The liposomes mainly accumulates in tumour, liver, spleen, and lymphatic

FIG. 2. Tumour growth delay assay of mouse RL xenograft model treated with liposomal G-CSF.

FIG. 3. Tumour growth delay assay of mouse RL xenograft model treated with liposomal G-CSF.

FIG. 4.

Tumour growth delay assay of mouse BT474 xenograft model (beast cancer) treated with liposomal G-CSF.

### EXAMPLES

### Example 1

### Preparation of DOPE Based Liposomes Comprising Cytokine

DOPE and DSPE-PEG 2000 were purchased from Genzyme Pharmaceuticals (Liestal, Switzerland). Cholesterol, HEPES, HSPC, TRITON-X100 (10% solution), sodium azide and sucrose were obtained from Sigma Aldrich. G-CSF was purchased from Chugai Pharmaceuticals (Granocyte™; lenograstim) or Teva Pharmaceuticals (Tevagrastim™; filgrastim).

G-CSF carrying liposomes (liposomal G-CSF) of different membrane composition were prepared using the thin film hydration method (Lasic 1993). Briefly, liposome components were dissolved in a chloroform/methanol mixture (9/1 v/v) at 60° C. and rotary evaporated to dryness under vacuum for 6 h. The resulting dried lipid films were hydrated with G-CSF (for concentrations, see batch table) dissolved in phosphate buffered saline (PBS; pH 7.4) solution for 2-6 h followed by three freeze-thaw cycles in a dry ice/acetone/methanol mixture and water, respectively. The liposomes at a lipid concentration of 30 mg/ml were extruded (Lipex extruder, Biomembrane Inc., Vancouver B.C., Canada) through Nucleopore polycarbonate filters with pore sizes of 800 nm (Nuclepore, West Chester, Pa., USA). The extruder, including filters, should be flushed with buffer comprising 2 ml/ml serum (e.g. BSA) before use. The lipid hydration, liposome extrusion and thawing process were performed above the gel-to-liquid-crystalline phase transition temperature of the phospholipids. For production of small sized liposomes the liposomes were downsized by stepwise extrusion through Nucleopore polycarbonate filters with pore sizes of 800, 400, 200, 100 and 80 nm.

Extraliposomal G-CSF may be removed by e.g. dialysis, diafiltration, or size exclusion chromatography, although this is not generally necessary. Dialysis was performed by placing disposable dialysers (MW cut off 100 000 D) containing the liposome dispersion, in a large volume of PBS solution (pH 7.4). The setup was protected from light and the dialysis ended when the trace of G-CSF in the dialysis was negligible. The liposome dispersion was then, until further use, stored in the fridge protected from light.

TABLE 1
Liposomal Cytokine Batches

| | | Conc. μg/ml | Lipid conc | Average size nm |
|---|---|---|---|---|
| Batch no. | Composition mol % | (cytokine) | mg/ml | (pdi) |
| 1 (218) | 62:8:30 (DOPE:DSPE- | 7.5 (lenograstim) | 30 | 246 (0.37) |
| | PEG2000:Chol) | | | |
| 2 (219) | 62:8:30 (DOPE:DSPE- | 0 | 30 | 504 (0.48) |
| | PEG2000:Chol) | | | |
| 3 (235) | 62:8:30 (DOPE:DSPE- | 7.5 (lenograstim) | 30 | 273 (0.34) |
| | PEG2000:Chol) | | | |
| 4 (236) | 62:8:30 (DOPE:DSPE- | 0 | 30 | 297 (0.4) |
| | PEG2000:Chol) | | | |
| 5 (252) | 62:8:30 mol % (DOPE:DSPE- | 7.5 (lenograstim) | 30 | 277 (0.37) |
| | PEG2000:Chol) | | | |
| 6 (253) | 62:8:30 mol % (DOPE:DSPE- | 7.5 (lenograstim) | 30 | 90 (0.06) |
| | PEG2000:Chol) | | | |
| 7 (254) | 57:6:38 mol % (HSPC:DSPE- | 7.5 (lenograstim) | 30 | 354 (0.18) |
| | PEG2000:Chol) | | | |
| 8 (255) | 57:6:38 mol % (HSPC:DSPE- | 7.5 (lenograstim) | 30 | 89 (0.04) |
| | PEG2000:Chol) | | | |
| 9 (256) | 62:8:30 mol % (DOPE:DSPE- | 7.5 (filgrastim) | 30 | 368 (0.29) |
| | PEG2000:Chol) | | | |
| 10 (262) | 62:8:30 mol % (DOPE:DSPE- | 45.3 (filgrastim) | 30 | 347 (0.50) |
| | PEG2000:Chol) | | | |
| 11 (263) | 62:8:30 mol % (DOPE:DSPE- | 60 (filgrastim) | 30 | 180 (0.28) |
| | PEG2000:Chol) | | | |
| 12 (283) | 52:8:40 mol % (DOPE:DSPE- | 10 (lenograstim) | 30 | 371 (0.30) |
| | PEG 2000:Chol) | | | |
| 13 (264) | DOPE:DSPE-PEG | Filgrastim | 30 | 111 (0.1) |
| | 2000:Chol 62:8:30 | 80 ug/ml | | |
| 14 (270) | DOPE:DSPE-PEG | Filgrastim | | 207 (0.36) |
| | 2000:Chol 62:8:30 mol % | 40 ug/ml | | |
| 15 (271) | DOPE:DSPE-PEG | Filgrastim | | 122 (0.06) |
| | 2000:Chol 62:8:30 | 40 ug/ml | | |
| 16 (272) | DOPE:DSPC:DSPE-PEG | Lenograstim | | 369 (0.31) |
| | 2000:Chol 62:10:8:20 | 10 ug/ml | | |
| 17 (273) | DOPE:DSPC:DSPE-PEG | Lenograstim | | 373 (0.51) |
| | 2000:Chol 62:10:8:20 | 40 ug/ml | | |
| 18 (274) | DOPE:DSPC:DSPE-PEG | Lenograstim | | 91.7 (0.07) |
| | 2000:Chol 62:10:8:20 | 40 ug/ml | | |
| 19 (283) | DOPE:DSPE-PEG | Lenograstim | | 371 (0.30) |
| | 2000:Chol 52:8:40 | 10 ug/ml | | |
| 20 (311) | DOPC:DSPE-PEG | Lenograstim | | 124 (0.084) |
| | 2000:Chol 62:8:30 | 10 ug/ml | | |
| 21 (312) | DOPS:DSPE-PEG | Lenograstim | | 131 (0.056) |
| | 2000:Chol 62:8:30 | 10 ug/ml | | |
| 22 (313) | DOPE:LysoPE:DSPE-PEG | Lenograstim | | 136 (0.084) |
| | 2000:Chol 52:10:8:30 | 10 ug/ml | | |
| 23 (314) | DOPE:DSPE-PEG | Lenograstim ≈ | | 192 (0.253) |
| | 2000:DSPE-PEG-MAL:Chol | 30 ug/ml | | |
| | 62:8:0,03:30 | | | |
| 24 (315) | DOPE:DSPE-PEG | Lenograstim ≈ | | 200 (0.258) |
| | 2000: DSPE-PEG-MAL:Chol | 30 ug/ml | | |
| | 62:8:0.03:30 | | | |
| 25 (316) | DSPE-PEG 2000:DSPE- | Lenograstim ≈ | | |
| | PEG-MAL 62:8:0.03:30 | 10 ug/ml | | |
| 26 (317) | DOPE:DSPC:DSPE-PEG | Lenograstim ≈ | | 127 (0.126) |
| | 2000:DSPE-PEG-MAL:Chol | 30 ug/ml | | |
| | 62:10:8:0.1:20 | | | |
| 27 (322) | 62:8:30 (DOPE:DSPE- | Granocyte | 30 | 188 (0.26) |
| | PEG2000:Chol) | 10 ug/ml | | |
| 28 (328) | DOPE:DSPE-PEG | Lenograstim | | 128 (0.172) |
| | 2000:Chol 54:16:30 | 10 ug/ml | | 108 (0.167) |
| 29 (330) | DOPE:DSPE-PEG | Lenograstim | | 233 (0.259) |
| | 5000:Chol 62:8:30 | 10 ug/ml | | 206 (0.253) |
| 30 (331) | DOPE:DSPE-PEG | Lenograstim | | 603 (0.405) |
| | 2000:Chol 58:12:30 | 10 ug/ml | | |

### Example 2

### Characterisation of Liposomal G-CSF

Liposomes were characterised with respect to key physicochemical properties like particle size and osmolality by use of well-established methodology.

The average particle size (intensity weighted) and size distribution were determined by photon correlation spectroscopy (PCS) at a scattering angle of 173° and 25 deg C. (Nanosizer, Malvern Instruments, Malvern, UK). The width of the size distribution is defined by the polydispersity index. Prior to sample measurements the instruments was tested by running a latex standard (60 nm). For the PCS measurements, 5 μL of liposome dispersion (lipid conc. 30 mg/ml) was diluted with 2 mL sterile filtered isosmotic PBS solution (pH 7.4). Duplicates were analysed.

Osmolality was determined on non-diluted liposome dispersions by freezing point depression analysis (Fiske 210 Osmometer, Advanced Instruments, MA, US). Prior to sample measurements, a reference sample with an osmolality of 290 mosmol/kg was measured; if not within specifications, a two-step calibration was performed. Duplicates of liposome samples were analysed.

### Example 3

### RL Cell Line and Culture

The RL cell line, derived from a human transformed FL sample, was purchased and used as a model of Non-Hodgkin's Lymphoma (NHL) expressing CD20 antigen. Cells were maintained in culture medium consisting of RPMI-1640 (Life Technologies), 10% of fetal calf serum (Integro), 100 units/mL of penicillin and 100 mg/mL of streptomycin (Life Technologies). All cells were cultured at 37° C. in a 5% CO2 atmosphere.

Six-week-old female CB17 severe combined immune-deficient mice (SCID) mice purchased from Charles River laboratories (l′Arbresle) were bred under pathogen-free conditions at the animal facility of our institute. Animals were treated in accordance with the European Union guidelines and French laws for the laboratory animal care and use. The animals were kept in conventional housing. Access to food and water was provided ad libitum. This study was approved by the local animal ethical committee.

For lymphoma xenograft experiments, 1×10⁶ RL cells were injected subcutaneously on day 1. Mice were randomized into study groups when tumour volume was approximately 200 mm³.

A549 Her2 positive xenografts were surgically implanted and animals were randomized into study groups at a tumour size of approximately 20 mm³.

Animals were weighed and the tumour size was measured twice a week with an electronic calliper. The tumor volume (TV) was estimated from two dimensional tumor measure-ments by the formula: tumour volume (mm³) ¼ length (mm) width2/2. Median tumor growth inhibition (% TGI) was calculated according to the NCl formula: 1 ([TVtreated (day 34 20) 100/TVcontrol (day 34 20) 100]).

### Example 4

### In Vivo Studies

Six-week-old female CB17 severe combined immune-deficient mice (SCID) mice purchased from Charles River laboratories (l'Arbresle) were bred under pathogen-free conditions at the animal facility of our institute. Animals were treated in accordance with the European Union guidelines and French laws for the laboratory animal care and use. The animals were kept in conventional housing. Access to food and water was provided ad libitum. This study was approved by the local animal ethical committee. For xenograft experiments, 1×10⁶ RL cells were injected subcutaneously on day 1. Mice were randomized when tumour volume was approximately 200 mm³ in groups of 5 animals treatment was initiated. The mice were injected intravenously in the tail vein, once a week. They were weighed and the tumour size was measured twice a week with an electronic calliper. The tumour volume (TV) was estimated from two dimensional tumour measurements by the formula: tumour volume (mm³) ¼ length (mm) width2/2. Median tumour growth inhibition (% TGI) was calculated according to the NCl formula: 1 ([TVtreated (day 34 20) 100/TVcontrol (day 34 20) 100]).

### Example 5

### Biodistribution of Liposomes Comprising Unsaturated Phoshatidylethanolamine

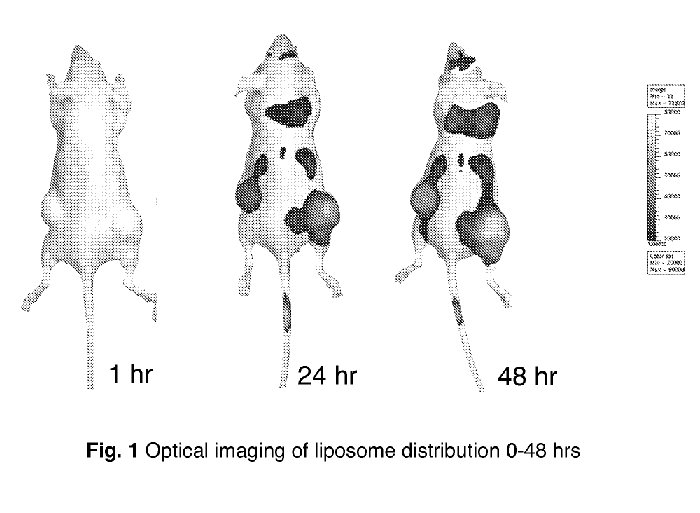

FIG. 1 shows distribution of DOPE liposomes as analysed by optical imaging. The liposomes were labelled with the florescent probe 1,1′-dioctadecyl-3,3,3′,3′-tetramethylindodicarbocyanine,4-chlorobenzenesulfonate salt (DiD; Molecular Probes, Eugene, Oreg.). Biodistribution assays will quantitively show that the liposome predominantly accumulates in liver and lymphatic tissue like spleen and lymph nodes of the head and neck.

### Example 6

### Therapy Study Results: Liposomal G-CSF Delays a Non-Hodgkin's Lymphoma Model

Six SCID mice carrying the RL tumour (see above) was randomized into two study groups: (1) untreated control and (2) liposomal G-CSF (Lipo-G; Batch #1). Lipo-G was administered intravenously (IV) in the tail vein at a total G-CSF dose of 37.5 μg/kg (100 μl injection volume). The injections were performed once a week for a duration of four weeks.

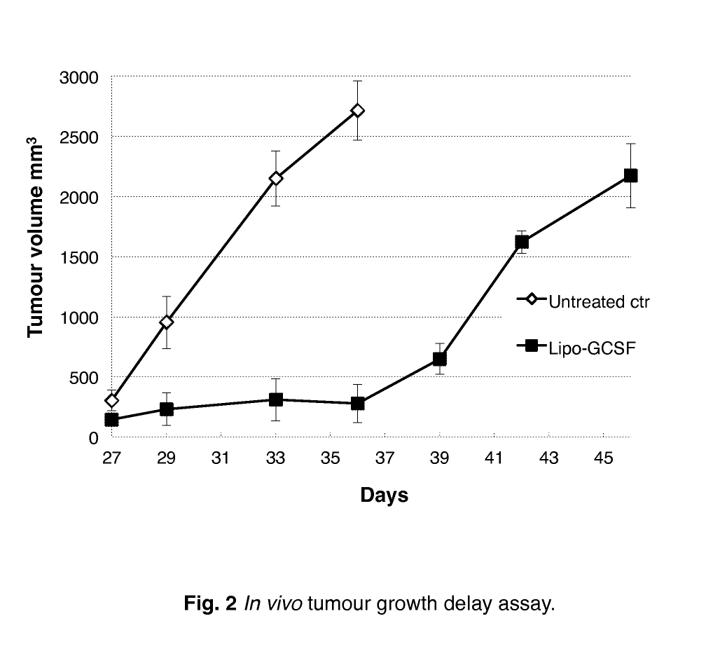

Tumour measurements were performed by caliper measurement of the diameter of the tumour (see above). Data represents the median with standard error mean. Untreated groups showed rapid tumour growth and were sacrificed after 29 days due to size of tumour. Administration of Lipo-G (liposomal G-CSF) led to a reduction in rate of tumour growth. See FIG. 2 for data presentation.

### Example 7

### Controlled Therapy Study: Liposomal G-CSF Is Superior to Free G-CSF

Fifteen SCID mice carrying the RL tumour (see above) was randomized into 4 study groups: (1) untreated control, (2) Empty liposome (Batch #4), (3) Free GCSF (lenograstim; Granocyte™), and (4) liposomal G-CSF (Lipo-GCSF; Batch #3). Empty liposomes were injected IV in the tail vein at a lipid dose of 30 mg/ml (lipid dose equivalent to Lipo-GCSF group) once weekly, lenograstim (Free GCSF) was administered intraperitoneally (IP) at a dose of 300 μg/kg three times per week (total weekly dose of 900 μg/kg), while liposomal G-CSF (Lipo-GCSF) was administered IV (tail vein) once weekly at a total G-CSF dose (unencapsulated and encapsulated C-CSF) of 37.5 μg/kg (100 pl injection volume). All regimens lasted for 4 weeks.

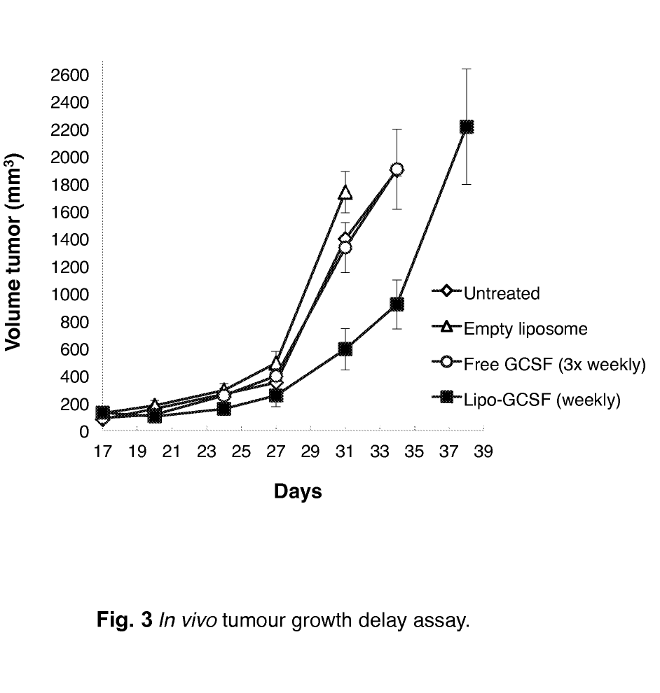

Tumour measurements were performed by caliper measurement of the diameter of the tumour (see above). Data represents the median with standard error mean. All controls showed rapid tumour growth, while the group receiving liposomal G-CSF showed delayed tumour growth. See FIG. 3 for data presentation. It is interesting to note that although the weekly dose of liposomal G-CSF is 24× lower than the free G-CSF, the latter group is still outperformed by the liposomal G-CSF group.

### Example 8

### Therapy Study Results: Liposomal G-CSF Delays a Breast Cancer Model

Six SCID mice carrying the BT474 tumour (human breast cancer model) was randomized into two study groups: (1) untreated control and (2) liposomal G-CSF (Lipo-G; Batch #27). Lipo-G was administered intravenously (IV) in the tail vein at a total G-CSF dose of 50 μg/kg (100 μl injection volume). The injections were performed once a week for a duration of two weeks.

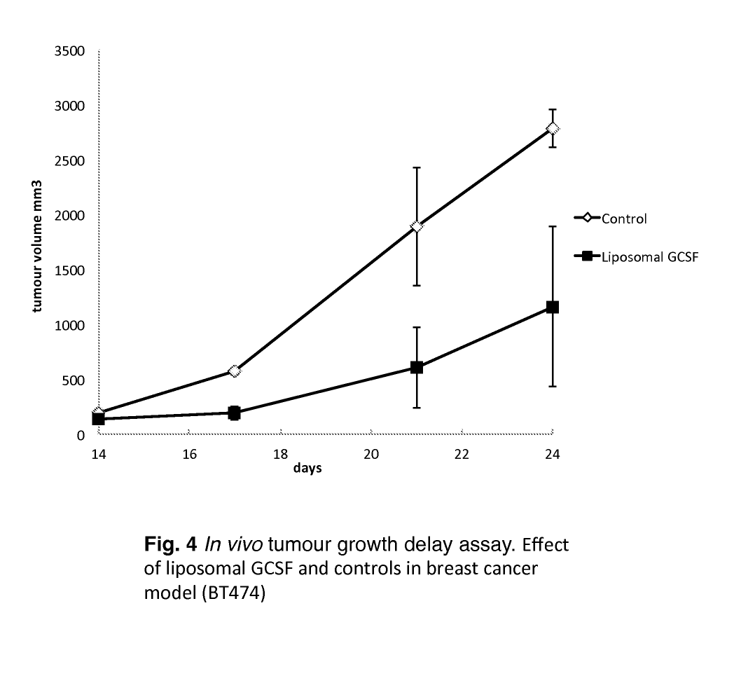

Tumour measurements were performed by caliper measurement of the diameter of the tumour (see above). Data represents the median with standard error mean. Untreated groups showed rapid tumour growth and were sacrificed after 24 days due to size of tumour. Administration of Lipo-G (liposomal G-CSF) led to a reduction in rate of tumour growth. See FIG. 4 for data presentation.

### REFERENCES

Anderson, P. M., D. C. Hanson, D. E. Hasz, M. R. Halet, B. R. Blazar and A. C. Ochoa (1994). “Cytokines in Liposomes: Preliminary Studies with Il -1, IL-2, Il-6, Gm-Csf and Interferon-Gamma.” Cytokine 6 (1): 92-101.

Anderson, P. M., E. Katsanis, A. S. Leonard, D. Schow, C. M. Loeffler, M. B. Goldstein and A. C. Ochoa (1990). “Increased Local Antitumor Effects of Interleukin 2 Liposomes in Mice with Mca-106 Sarcoma Pulmonary Metastases.” Cancer Res 50 (6): 1853-1856.

Cabanes, A., S. Even-Chen, J. Zimberoff, Y. Barenholz, E. Kedar and A. Gabizon (1999). “Enhancement of Antitumor Activity of Polyethylene Glycol-Coated Liposomal Doxorubicin with Soluble and Liposomal Interleukin 2.” Clin Cancer Res 5 (3): 687-693.

Debs, R. J., H. J. Fuchs, R. Philip, E. N. Brunette, N. Duzgunes, J. E. Shellito, D. Liggitt and J. R. Patton (1990). “Immunomodulatory and Toxic Effects of Free and Liposome-Encapsulated Tumor Necrosis Factor Alpha in Rats.” Cancer Res 50 (2): 375-380.

Honsik, C. J., G. Jung and R. A. Reisfeld (1986). “Lymphokine-Activated Killer Cells Targeted by Monoclonal Antibodies to the Disialogangliosides Gd2 and Gd3 Specifically Lyse Human Tumor Cells of Neuroectodermal Origin.” Proc Natl Acad Sci USA 83 (20): 7893-7897.

Kedar, E., E. Braun, Y. Rutkowski, N. Emanuel and Y. Barenholz (1994a). “Delivery of Cytokines by Liposomes. Ii. Interleukin-2 Encapsulated in Long-Circulating Sterically Stabilized Liposomes: Immunomodulatory and Anti-Tumor Activity in Mice.” J Immunother Emphasis Tumor Immunol 16 (2): 115-124.

Kedar, E., H. Gur, I. Babai, S. Samira, S. Even-Chen and Y. Barenholz (2000). “Delivery of Cytokines by Liposomes: Hematopoietic and Immunomodulatory Activity of Interleukin-2 Encapsulated in Conventional Liposomes and in Long-Circulating Liposomes.” J Immunother 23 (1): 131-145.

Kedar, E., O. Palgi, G. Golod, I. Babai and Y. Barenholz (1997). “Delivery of Cytokines by Liposomes. Iii. Liposome-Encapsulated Gm-Csf and Tnf-Alpha Show Improved Pharmacokinetics and Biological Activity and Reduced Toxicity in Mice.” J Immunother 20 (3): 180-193.

Kedar, E., Y. Rutkowski, E. Braun, N. Emanuel and Y. Barenholz (1994b). “Delivery of Cytokines by Liposomes. I. Preparation and Characterization of Interleukin-2 Encapsulated in Long-Circulating Sterically Stabilized Liposomes.” J Immunother Emphasis Tumor Immunol 16 (1): 47-59.

Kushner, B. H. and N. K. Cheung (1989). “Gm-Csf Enhances 3f8 Monoclonal Antibody-Dependent Cellular Cytotoxicity against Human Melanoma and Neuroblastoma.” Blood 73 (7): 1936-1941.

Lasic, D. D. (1993). Liposomes from Physics to Applications. Amsterdam, Amsterdam Elsevier Science Publishers BV.

Meyer, J., L. Whitcomb and D. Collins (1994). “Efficient Encapsulation of Proteins within Liposomes for Slow Release in Vivo.” Biochem Biophys Res Commun 199 (2): 433-438.

Munn, D. H. and N. K. Cheung (1987). “Interleukin-2 Enhancement of Monoclonal Antibody-Mediated Cellular Cytotoxicity against Human Melanoma.” Cancer Res 47 (24 Pt 1): 6600-6605.

Ottonello, L., A. L. Epstein, P. Dapino, P. Barbera, P. Morone and F. Dallegri (1999). “Monoclonal Lym-1 Antibody-Dependent Cytolysis by Neutrophils Exposed to Granulocyte-Macrophage Colony-Stimulating Factor: Intervention of Fcgammarii (Cd32), Cd11b-Cd18 Integrins, and Cd66b Glycoproteins.” Blood 93 (10): 3505-3511.

Rourke, A. M., Y. Cha and D. Collins (1996). “Stabilization of Granulocyte Colony-Stimulating Factor and Structurally Analogous Growth Factors by Anionic Phospholipids.” Biochemistry 35 (36): 11913-11917.

Stockmeyer, B., D. Elsasser, M. Dechant, R. Repp, M. Gramatzki, M. J. Glennie, J. G. van de Winkel and T. Valerius (2001). “Mechanisms of G-Csf- or Gm-Csf-Stimulated Tumor Cell Killing by Fc Receptor-Directed Bispecific Antibodies.” J Immunol Methods 248 (1-2): 103-111.

U.S. Pat. No. 5,225,212

van der Kolk, L. E., M. de Haas, A. J. Grillo-Lopez, J. W. Baars and M. H. van Oers (2002). “Analysis of Cd20-Dependent Cellular Cytotoxicity by G-Csf-Stimulated Neutrophils.” Leukemia 16 (4): 693-699.

WO 2009/075582

WO 2010/143969

WO 2010/143970

WO 96/29989

We claim:

1. A liposome comprising a colony stimulating factor (CSF), polyethylene glycol (PEG) or a derivate thereof, and an unsaturated phospholipid selected from the group consisting of phoshatidylethanolamine (PE) and phosphatidylserine (PS) for use in treatment of cancer, wherein said liposome is not activated by acoustic energy or ultrasound.

2. The liposome of claim 1, wherein the phospholipid has an acyl chain comprising at least 16 carbon atoms.

3. The liposome of claim 1, wherein the phospholipid has a acyl chain comprising at least 18 carbon atoms.

4. The liposome of claim 1, where the PS is 1,2-dioleoyl-sn-glycero-3-phospho-L-serine (DOPS), 1-stearoyl-2-oleoyl-sn-glycero-3-phospho-L-serine (SOSE), or a combination thereof.

5. The liposome of claim 1, wherein the phospholipid or PE is 1,2-dioleoyl-sn-glycero-3-phosphoethanolamine (DOPE) and/or 1-stearoyl-2-oleoyl-sn-glycero-3-phosphoethanolamine (SOPE).

6. The liposome of claim 1, wherein the phospholipid or PE is 1,2-dioleoyl-sn-glycero-3-phosphoethanolamine (DOPE).

7. The liposome of claim 5, wherein the PE or DOPE concentration is within the range 32 to 75 mol %.

8. The liposome of claim 5, wherein the PE or DOPE concentration is at least 40 mol %.

9. The liposome of claim 5, wherein the PE or DOPE concentration is at least 50 mol %.

10. The liposome of claim 1, wherein the PEG is 1,2-distearoyl-sn-glycero-3-phosphoethanolamine-N-[methoxy(polyethylene glycol)-2000] (DSPE-PEG2000).

11. The liposome of claim 1, wherein the PEG concentration is at least 8 mol %.

12. The liposome of claim 1, wherein said liposome has an average diameter within the range 50 nm to 1200 nm.

13. The liposome of claim 1, wherein the liposome has an average diameter within the range 80-400 nm.

14. The liposome of claim 1, wherein the liposome further comprises cholesterol.

15. The liposome of claim 1, wherein the liposome comprises an immunomodulator and DOPE:PEG:CHOL at molar percentages 62:8:30.

16. The liposome of claim 1, wherein the CSF is granulocyte monocyte-colony stimulating factor (GM-CSF), granulocyte-colony stimulating factor (G-CSF), or monocyte-colony stimulating factor (M-CSF).

17. The liposome of claim 1, wherein the CSF is granulocyte-colony stimulating factor (G-CSF).

18. The liposome of claim 1, wherein the CSF or G-CSF is filgrastim or lenograstim.

19. The liposome of claim 1, wherein the cancer is lymphoma, breast cancer, or colorectal cancer.
